(19) United States
(12) Patent Application Publication
(10) Pub. No.: US 2017/0370849 A1
(43) Pub. Date: Dec. 28, 2017

(54) METHODS TO IDENTIFY CANDIDA SPECIES FROM PATIENT CULTURES USING RAMAN SPECTROMETRY

(71) Applicant: Nitin Shashikant CHOUTHAI, NOVI, MI (US)

(72) Inventor: Nitin Shashikant CHOUTHAI, NOVI, MI (US)

(21) Appl. No.: 15/524,769

(22) PCT Filed: Dec. 11, 2015
(86) PCT No.: PCT/IN2015/000447

§ 371 Date: May 5, 2017

Related U.S. Application Data
(60) Provisional application No. 62/091,341, filed on Dec. 12, 2014.

(51) Int. Cl. G01N 21/65; C12Q 1/04; G01N 21/31; G01N 21/3577

(52) U.S. Cl. CPC G01N 21/65; C12Q 1/04; G01N 21/31; G01N 2021/655; G01N 21/3577

(57) ABSTRACT

Methods to identify fungal or bacterial species using Raman spectrometry, among other techniques are described. The identification can occur from samples taken directly from cultures grown from a patient sample. The methods significantly decrease the time required to identity a fungal or bacterial species in a clinical setting, allowing more effective treatments, especially in low birth weight neonates and immunocompromised individuals.

### FIELD OF THE DISCLOSURE

The current disclosure provides methods to identify fungal or bacterial species using Raman spectrometry, among other techniques. The identification can occur from samples taken directly from cultures grown from a patient sample. The methods significantly decrease the time required to identity fungal or bacterial species in a clinical setting, allowing more effective treatments of infections, especially in low birth weight neonates and immunocompromised individuals.

### BACKGROUND OF THE DISCLOSURE

Invasive Candidiasis is a life threatening infection which mainly affects individuals with serious underlying co-morbidities. Infections usually occur in the hospital intensive care unit (ICU) where it is the fourth most common bloodstream infection. The disease disproportionally effects minorities as a higher incidence has been observed among Blacks/African-Americans and babies less than one month old. It is estimated that between 5% and 20% of newborns that weigh less than 1000 grams (2.2 pounds) at birth develop invasive candidiasis. In the US, about 1.5% of newborns are born with very low birth weights.

There are different Candida species; however, more than 90% of invasive infections are caused by Candida albicans, Candida glabrata, Candida parapsilosis, Candida tropicalis and Candida krusei. The most effective therapy for each of these species can differ and the wrong treatment may destroy commensurable Candida, allowing the pathogenic Candida to flourish; so it is important to identify the proper strain and align the proper treatment.

To test for species of Candida currently, dilutions of patient blood, plasma or serum are diluted and plated onto culture dishes and allowed to grow into colonies. After 48 to 72 hours, a fungal colony is selected and used to culture a number of agar plates using various and different types of media. After an additional 24 to 72 hours, the species can be identified depending on the types of media the Candida does or does not grow upon. In some instances, patients do not survive the duration of this testing time.

### SUMMARY OF THE DISCLOSURE

The currently disclosed methods allow the rapid identification of Candida species after the first culture, when a colony is isolated without having to perform subsequent cultures. This advance significantly shortens the time to a species diagnosis, allowing intervention with appropriate and tailored therapeutics, saving more patients from the harmful effects of Candida infections. The currently disclosed methods achieve this benefit by identifying Candida species following the first culture using an analysis such spectral analysis (e.g., Raman spectroscopy).

### BRIEF DESCRIPTION OF THE FIGURES

FIG. 1 shows an example of Stimulated Raman Scattering (SRS) measurement of a sample according to conventional schemes.

FIG. 2 shows mean normalized curves of the Raman signatures of five Candida species represented individually as well as compiled together.

FIG. 3 shows compiled mean normalized curves of Raman signatures of unknown Candida species.

FIG. 4 shows compiled mean normalized curves of Raman signatures of unknown Candida species.

### DETAILED DESCRIPTION

Invasive Candidiasis is a life threatening infection which mainly affects individuals with serious underlying co-morbidities. Infections usually occur in the hospital intensive care unit (ICU) where it is the fourth most common bloodstream infection. The disease disproportionally effects minorities as a higher incidence has been observed among Blacks/African-Americans and babies less than one month old.

There are different Candida species; however, more than 90% of invasive infections are caused by Candida albicans, Candida glabrata, Candida parapsilosis, Candida tropicalis and Candida krusei. The most effective therapy for each of these species can differ and the wrong treatment may destroy commensurable Candida, allowing the pathogenic Candida to flourish; so it is important to identify the proper strain and align the proper treatment.

To test for the species of Candida currently, dilutions of patient blood, plasma or serum are diluted and plated onto culture dishes and allowed to grow into colonies. After 48 to 72 hours, a fungal colony is selected and used to culture a number of agar plates using various and different types of media. After an additional 24 to 72 hours, the species can be identified depending on the types of media the Candida does or does not grow upon. In some instances, patients do not survive the duration of this testing time.

The currently disclosed methods allow the rapid identification of Candida species after the first culture, when a colony is isolated without having to perform subsequent cultures. This advance significantly shortens the time to a species diagnosis, allowing intervention with appropriate and tailored therapeutics, saving more patients from the harmful effects of Candida infections.

The currently disclosed methods achieve this benefit by identifying Candida species following the first culture using methods such as spectral analysis methods (e.g., Raman spectroscopy). This advance has numerous advantages including that species identification can occur within minutes after primary culture, 24 to 72 hours faster than currently-available methods, a factor that is critical in neonatal care.

As used herein, spectral analysis methods can include any type of analyses that assess atomic bond vibrations to generate characteristic spectra or “fingerprints” of matrices, cells, proteins and other organic substances. Spectral analysis methods also include diffuse reflectance spectroscopy, dual photo fluorescence, fluorescence spectroscopy, infrared spectroscopy, phosphorescence, Raman spectroscopy, resonance Raman spectroscopy, Spatially Offset Raman spectroscopy (SORS), Surface Enhanced Raman Spectroscopy (SERS), transmission and absorbance spectroscopy, transmission Raman spectroscopy, terahertz spectroscopy, and X-ray fluorescence. Mass spectrometry techniques can also be used. Exemplary mass spectrometry techniques include desorption electrospray ionization (DESI) mass spectrometry, electrospray ionization (ESI) mass spectrometry, gas chromatography (GC) mass spectrometry, inlet ionization mass spectrometry, laser spray ionization (LSI) mass spectrometry, liquid chromatography (LC) mass spectrometry, matrix-assisted laser desorption ionization time of flight (MALDI-TOF) mass spectrometry, and selected ion flow tube (SIFT) spectrometry. Other forms of characterization such as epifluorescence, reflectance, absorbance, and/or scatter measurements, can also be employed used. Samples can also be characterized using more than one of the above-described methods. In these embodiments, sample characterization can be carried out sequentially or simultaneously.

The sample illumination source, or excitation source, can be selected from any number of suitable light sources as known to those skilled in the art. Any portion of the electromagnetic spectrum that produces usable data can be used. Light sources capable of emission in the ultraviolet, visible and/or near-infrared spectra, as well as other portions of the electromagnetic spectrum, can be utilized. For example, light sources can be a deuterium or xenon arc lamp for generation of ultraviolet light and/or a tungsten halogen lamp for generation of visible/near-infrared excitation. Alternatively, a plurality of narrowband light sources, such as light emitting diodes and/or lasers, may be spatially and/or temporally multiplexed to provide a multi-wavelength excitation source. For example, light emitting diodes are available from 240 nm to in excess of 900 nm and the sources have a spectral bandwidth of 20-40 nm (full width at half maximum).

The spectral selectivity of any of the excitation source can be improved by using spectral discrimination methods such as a scanning monochromator. Other methods of discrimination such as acousto-optic tunable filters, liquid crystal tunable filters, an array of optical interference filters, prism spectrographs, etc., can also be used individually or in any combination.

Emission from an excited sample can be measured by using suitable methods of spectral discrimination, in particular embodiments with a spectrometer. The spectrometer can be a scanning monochromator that detects specific emission wavelengths whereby the output from the monochromator is detected by a photomultiplier tube and/or the spectrometer can be configured as an imaging spectrograph whereby the output is detected by an imaging detector array such as a charge-coupled device (CCD) detector array. In one embodiment, a discriminator allows the observation of the fluorescence and/or scattering signal by a photodetection method (e.g., a photomultiplier tube, avalanche photodiode, CCD detector array, and/or electron multiplying charge coupled device (EMCCD) detector array).

Particular embodiments disclosed herein utilize Raman spectroscopy. Raman spectroscopy relies on the Raman scattering, of monochromatic light, usually from a laser in the visible, near infrared, or near ultraviolet range. The laser light interacts with molecular vibrations, photons or other excitations in the sampled system, resulting in the energy of the laser photons being shifted up or down. The shift in energy gives information about the vibrational modes in the sampled system. Raman spectroscopy is commonly used in chemistry to provide a fingerprint by which molecules can be identified.

More, particularly, when a probe beam of radiation described by an electric field (E) interacts with a sample, a dipole moment (μ) is induced in the molecules of the sample as follows: [μ=a×E] where a is the polarizability of the molecule. The polarizability is a proportionality constant describing the deformability of the molecule.

For a sample to be evaluated by Raman spectroscopy, it must include molecules having molecular bonds with a polarizability that varies as a function of interatomic distance. Light striking a molecule with such a bond can be absorbed and re-emitted at a different frequency (Raman-shifted), corresponding to the frequency of the bond's vibrational mode. If the molecule is in its ground state upon interaction with the probe beam, light can be absorbed and re-emitted at a lower frequency (Stokes-shifted Raman scattering). If the molecule is in a vibrationally excited state when it interacts with the probe beam, the interaction can cause the molecule to give up its vibrational energy to the probe beam and drop to the ground state (anti-Stokes Raman scattering). Light absorbed and re-emitted at the same frequency is known as Rayleigh, or elastic scattering. See Omega Optical (Brattleboro, Vt.) Product Descriptions for Further Information.

Raman spectroscopy is becoming an increasingly practical technique to analyse biological samples because of its minimal sample preparation requirements and compatibility with biological materials in aqueous solutions (Lawson et al., J. Raman Spectrosc. 28, 111 (1997); Krafft, Anal. Bioanal. Chem. 378, 60 (2004)). Surface enhanced resonance Raman spectroscopy (SERRS) provides excellent enhancement, making it an attractive technique for applications in protein, nucleic acid, and related biomarker analysis (Kneipp et al., Chem. Rev. 99, 2957 (1999)). SERRS not only overcomes the gap between the inherent sensitivity of Raman scattering and fluorescence, but the Raman spectral features are also larger (and sharper) than fluorescence from the same chromophore (Campion and Kambhampati, Chem. Soc. Rev. 27, 241 (1998)).

The Stimulated Raman Scattering (SRS) process can be employed for high-speed vibrational imaging. SRS provides strong Raman signaling and exhibits no non-resonant background. SRS is a third order nonlinear optical process, which involves two laser fields, namely a pump field at ωₚ and a Stokes field at ω_{S}. When the beating frequency (ωₚ−ω_{S}) is tuned to excite a molecular vibration, the energy difference between ωₚ and ω_{S} pumps the molecule from a ground state to a vibrationally excited state. The laser field manifests this as a weak decrease of pump beam intensity, called stimulated Raman loss (SRL), and corresponding increase of Stokes beam intensity, called stimulated Raman gain (SRG). Using heterodyne detection, SRS is able to offer quantitative spectral information with a pixel dwell time of few μs.

To measure weak laser intensity changes ΔIₚ, e.g., on the order of 0.01% or smaller, a heterodyne detection approach can be used. In the case of SRL, the Stokes beam intensity Is modulated and the pump beam intensity Iₚ is recorded by a photodiode. The induced modulation is then extracted by a lock-in amplifier. Theoretically, the modulation depth induced by SRL, I_{SRL}/Iₚ, is linearly proportional to the Raman cross section, a, molar concentration of the target molecule, N, and the Stokes beam intensity, i.e.: [I_{SRL}/Iₚ ∝σ N I_{S}].

A megahertz (MHz) modulation rate can be used to reduce effects of low frequency laser noise. Lock-in amplifiers (analog or digital) are commonly used for extraction of heterodyne-detected signals like SRS. So far, fast SRS imaging is mostly implemented by narrowband laser excitation of single isolated Raman band.

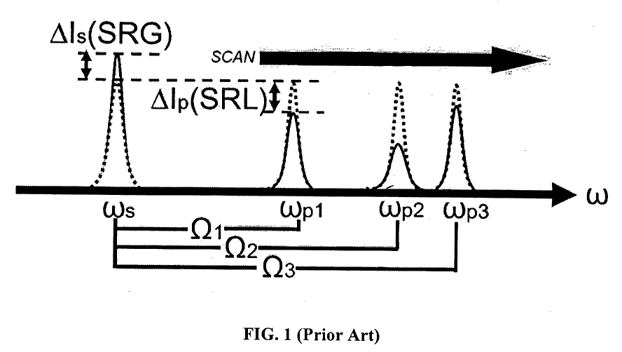

FIG. 1 shows an example of SRS measurement of a sample according to conventional schemes. The angular frequency ωₚ of a narrowband pump beam is scanned and the angular frequency ω_{S} of a narrowband Stokes beam is held fixed. With ωₚ−ω_{S} tuned to a molecular vibration at frequency Ωₙ, nε[1, 2, 3], the pump beam intensity is slightly decreased by stimulated Raman loss (SRL; ΔI^{p}) and the Stokes beam intensity is slightly increased by stimulated Raman gain (SRG; ΔI_{S}). Only parts of the spectrum at which SRL occurs are illustrated here. Dashed lines show the incident radiation before interaction with the sample; solid lines show the radiation resulting from interaction with the sample. For further information, see WO2013/110023, International Application No. PCT/US2013/022348, and U.S. Pat. Nos. 6,809,814, and 6,108,081.

One embodiment disclosed herein includes a Raman spectroscopy method of identifying Candida species in a sample comprising: (a) establishing a database of characteristic curves of Candida species; (b) performing Raman spectrum testing analysis on a sample obtained from a subject and obtaining corresponding characteristic curves; and (c) searching the database of step (a) for a match to the characteristic curves obtained in step (b), and (d) identifying a species of Candida in the sample. Following identification, a therapeutically effective treatment for the subject can be directed.

In another embodiment, the methods include testing a sample obtained from a subject suspected of suffering from a Candida infection for the presence of a spectral curve associated with a Candida species. The testing can be done using Raman spectroscopy.

Another embodiment includes accumulating spectral data from a sample, obtained from a subject and identifying a spectral feature which is indicative of an infection state. Further embodiments include directing a treatment for the subject based on the identified spectral features.

Particular embodiments disclosed herein identify a species of Candida causing Candida infection in a subject. Exemplary Candida species that can be identified using the methods disclosed herein include Candida albicans, Candida dubliniensis, Candida famata, Candida glabrata, Candida guilliermondii, Candida kefyr, Candida krusei, Candida lambica, Candida lipolytica, Candida lustitaniae, Candida parapsilosis, Candida pseudotropicalis, Candida pulcherrima, Candida revkaufi, Candida rugosa, Candida tropicalis, Candida stellatoidea, Candida utilis, Candida viswanathii, Candida xestobii and any other Candida spp. yeast. Examples of particular strains include C. albicans (ATCC 28815), C. parapsilosis (ATCC 34136), C. glabrata (ATCC 2001), C. krusei (ATCC 6258), C. tropicalis (ATCC 13803), sAA001 (ATCC20336), sAA002 (ATCC20913), sAA003 (ATCC20962), sAA496 (US2012/0077252), sAA106 (US2012/0077252), SU-2 (ura3−/ura3−), and H5343 (beta oxidation blocked; U.S. Pat. No. 5,648,247).

The methods of the current disclosure could also be applied to species of other fungus such as: Absidia species (e.g., Absidia corymbifera and Absidia ramosa), Aspergillus species, (e.g., Aspergillus flavus, Aspergillus fumigatus, Aspergillus nidulans, Aspergillus niger, and Aspergillus terreus), Basidiobolus ranarum, Blastomyces dermatitidis), Coccidioides immitis, Conidiobolus species; Cryptococcus neoforms, Cunninghamella species, dermatophytes, Histoplasma capsulatum, Microsporum gypseum, Mucor pusillus, Paracoccidioides brasiliensis, Pseudallescheria boydii, Rhinosporidium seeberi, Pneumocystis carinii, Rhizopus species (e.g., Rhizopus arrhizus, Rhizopus oryzae, and Rhizopus microsporus), Saccharomyces species, Sporothrix schenckii, zygomycetes, and classes such as Zygomycetes, Ascomycetes, the Basidiomycetes, Deuteromycetes, and Oomycetes.

The methods of the current disclosure could also be applied to bacteria such as the Aquaspirillum family, Azospirillum family, Azotobacteraceae family, Bacteroidaceae family, Bartonella species, Bdellovibrio family, Campylobacter species, Chlamydia species (e.g., Chlamydia pneumoniae), clostridium, Enterobacteriaceae family (e.g., Citrobacter species, Edwardsiella, Enterobacter aerogenes, Erwinia species, Escherichia coli, Hafnia species, Klebsiella species, Morganella species, Proteus vulgaris, Providencia, Salmonella species, Serratia marcescens, and Shigella flexneri), Gardinella family, Haemophilus influenzae, Halobacteriaceae family, Helicobacter family, Legionallaceae family, Listeria species, Methylococcaceae family, mycobacteria (e.g., Mycobacterium tuberculosis), Neisseriaceae family, Oceanospirillum family, Pasteurellaceae family, Pneumococcus species, Pseudomonas species, Rhizobiaceae family, Spirillum family, Spirosomaceae family, Staphylococcuss (e.g., methicillin resistant Staphylococcus aureus and Staphylococcus pyrogenes), Streptococcus (e.g., Streptococcus enteritidis, Streptococcus fasciae, and Streptococcus pneumoniae), Vampirovibr Helicobacter family, and Vampirovibrio family.

As previously stated, following identification of a species causing infection in a subject, a therapeutically effective treatment can be directed. Subjects include humans (and in particular embodiments, low birth weight neonates), veterinary 1.5 animals (dogs, cats, reptiles, birds, hamsters, etc.) livestock (horses, cattle, goats, pigs, chickens, etc.), research animals (monkeys, rats, mice, fish, etc.) and other animals, such as zoo animals (e.g., bears, giraffe, elephant, lemurs).

Therapeutically effective amounts provide therapeutic treatments to subjects. A “therapeutic treatment” includes a treatment administered to a subject who displays symptoms or signs of an infection (e.g., Candida infection) and is administered to the subject for the purpose of diminishing or eliminating those signs or symptoms of infection. The therapeutic treatment can reduce, control, or eliminate the presence or activity of infection and/or reduce control or eliminate side effects of the infection.

Within the context of Candida infections, therapeutically effective amounts can decrease fungal infection load and can be evidenced by a negative blood culture result; microscopic testing of samples using, for example, a potassium hydroxide smear, Gram stain, or methylene blue; clearance of white blood cells (WBCs), red blood cells (RBCs), protein, and yeast cells from the urine; an endoscopic test; or a sign of clinical improvement related to one or more of the following non-limiting symptoms: sore and painful mouth; burning mouth or tongue; dysphagia; erythema; odynophagia; retrosternal pain; epigastric pain; nausea and/or vomiting; fever and chills; abdominal mass; thick, curdlike discharge from the vagina with a normal cervix upon speculum examination; penile pruritus; urinary frequency or urgency; dysuria; hematuria; suprapubic pain; intermittent urinary tract obstruction; anuria and/or renal insufficiency.

For administration, therapeutically effective amounts (also referred to herein as doses) for administration to a particular subject can be determined by a physician, veterinarian or researcher taking into account parameters such as physical and physiological factors including target, body weight, severity of infection, species causing infection, stage of infection, previous or concurrent therapeutic interventions, idiopathy of the subject and route of administration.

Useful doses can range from 0.1 to 5 μg/kg or from 0.5 to 1 μg/kg. In other non-limiting examples, a dose can include 1 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 950 μg/kg, 1000 μg/kg, 0.1 to 5 mg/kg or from 0.5 to 1 mg/kg. In other non-limiting examples, a dose can include 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 150 mg/kg, 200 mg/kg, 250 mg/kg, 350 mg/kg, 400 mg/kg, 450 mg/kg, 500 mg/kg, 550 mg/kg, 600 mg/kg, 650 mg/kg, 700 mg/kg, 750 mg/kg, 800 mg/kg, 850 mg/kg, 900 mg/kg, 950 mg/kg, 1000 mg/kg or more.

Therapeutically effective amounts can be achieved by administering single or multiple doses during the course of a treatment regimen (e.g., hourly, three times daily, twice daily, daily, every other day, every 3 days, every 4 days, every 5 days, every 6 days, weekly, every 2 weeks, every 3 weeks, monthly, every 2 months, every 3 months, etc.).

Administration can occur through any available route including injection, inhalation, infusion, perfusion, lavage or ingestion. Routes of administration can include intravenous, intradermal, intraarterial, intraparenteral, intranasal, intranodal, intralymphatic, intraperitoneal, intralesional, intraprostatic, intravaginal, intrarectal, topical, intrathecal, intramuscular, intravesicular, oral, subcutaneous, and/or sublingual administration. Administration can also occur by intradermal, intraarterial, intraparenteral, intranasal, intranodal, intralymphatic, intraperitoneal, intralesional, intraprostatic, intravaginal, intrarectal, topical, intrathecal, intramuscular, intravesicular, oral, subcutaneous, and/or sublingual injection.

Information derived from the methods disclosed herein can direct treatment options in subjects. For example, a treatment regimen for C. albicans, C. tropicalis, C. parapsilosis, C. kefyr, C. dubliniensis, C. lusitaniae, and C. guilliermondi includes a fluconazole loading dose, followed by, in certain embodiments, fluconazole at a dose of 400 mg/d for at least 2 weeks of therapy after a demonstrated negative blood culture result or clinical signs of improvement.

C. glabrata is resistant to fluconazole in 15%-25% of cases and has decreased susceptibility to most antifungals. Thus, C. glabrata infections require a change in conventional antifungal therapy. The drugs of choice for such infections are the echinocandins: caspofungin (in some embodiments, a loading dose followed by 50, mg/d; an anidulafungin loading dose, followed by 100 mg/d; or micafungin 100 mg/day intravenously). An alternative is voriconazole (in some embodiments, 6 mg/kg administered twice on the first day, followed by 3 mg/kg twice per day or 200 mg twice per day orally; other options include amphotericin B deoxycholate (1 mg/kg/d), or lipid preparations of amphotericin B at 3-5 mg/kg/d).

C. krusei infections should be treated with agent other than fluconazole because this organism is resistant to fluconazole and has decreased susceptibility to itraconazole, ketoconazole, and amphotericin B. Thus, echinocandins (caspofungin, anidulafungin, or micafungin) voriconazole, or amphotericin B are preferred, in some embodiments at 1 mg/kg/d. Infections due to C. lusitaniae or C. guilliermondi require the use of fluconazole, voriconazole, or the echinocandins because these isolates are often resistant to amphotericin B or develop resistance to amphotericin B during treatment.

As is understood by one of ordinary skill in the art, additional treatment options include Voriconazole (in some embodiments, 6 mg/kg intravenously or orally twice per day, followed by 3 mg/kg orally twice per day or 200 mg orally twice per day); Amphotericin B deoxycholate (in some embodiments, 0.7 mg/kg/d intravenously for a total dose of 1-2 g over a 4- to 6-week period, although for the treatment of invasive candidiasis caused by less-susceptible species, such as C. glabrata and C. krusei, higher doses (up to 1 mg/kg/d) should be considered).

Embodiments disclosed herein are particularly helpful in speeding the time of species identification in the treatment of low birth weight neonates (e.g., those weighing less than 5 pounds, 8 ounces at birth).

The Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### EXAMPLES

Late-onset sepsis occurs in 2-20% of extremely low birth weight neonates and up to 12% of these infections are due to Candida species (Stoll et al., Pediatrics 2002; 110(2 Pt 1): 285-291). Furthermore, Candida Blood Stream Infections (BSIs) are associated with a 25-40% mortality rate (Greenberg et al., J Pediatrics 2012; 161(2):264-269 e2). Invasive Candida infections have also been associated with 73% rate of neurodevelopmental impairment or death (Benjamin et al. Pediatrics 200; 117(1): 84-92). Currently, identification of yeasts grown in a blood culture takes 24-48 hr which may delay appropriate antifungal therapy (Telenti and Roberts, Eur J Clin Microbiol Infect Dis 1989; 8(9): 825-831; Matthews, J Antimicrob Chemother 1993; 31(6): 809-812). Raman Spectroscopy (RS) has been used to identify many characteristics of individual cell types including cancer tissue and stem cell types (Chan et al., Biophys J 2006; 90(2): 648-656; Kast et al., Biopolymers 2008; 89(3): 235-241; Leslie et al. Pediatr Neurosurg 2012; 48(2):109-117. Creating a reference library of specific signatures of clinically important pathogens may allow the use of spectroscopy as a tool for rapid identification of pathogens in clinical practice. Candida albicans (C. albicans) and Candida parapsilosis (C. parapsilosis) are the most common agents that cause fungal BSIs in neoantes (Fridkin et al., Pediatrics 2006 May; 117(5): 1680-1687. In addition, the incidence of Candida glabrata (C. glabrata) and Candida krusei (C. krusei) infections has increased due to increased use of fluconazole, to which these organisms are resistant (Fridkin, supra, Mean et al., Critical Care 2008; 12(1): 204. A small percentage of fungal BSIs are also caused by Candida Tropicalis (C. tropicalis) (Fridkin, supra; Mean et al, supra). The objective of the study disclosed herein was to establish signatures of C. albicans, C. parapsilosis, C. glabrata, C. krusei and C. tropicalis that have been grown on Sabouraud Dextrose Agar (SDA) using RS. A second objective was to determine if these signatures can be used to identify unknown Candida isolates.

Materials and Methods. Colonies from five commonly occurring Candida species grown on SDA media were evaluated. Candida species were obtained from American Type CultureCollection (C. albicans: ATCC 28815, C. parapsilosis: ATCC 34136, C. glabrata: ATCC 2001, C. krusei: ATCC 6258 and C. tropicalis: ATCC 13803). The identities of the samples were confirmed using the API 20C identification system. All culture plates were inoculated using standard techniques of equal amount of inoculum and incubated for 24 hr. The plates with growth of fungi were directly placed under Raman spectroscope without additional processing. All spectral measurements (C. albicans: 115 spectra, C. parapsilosis: 112 spectra, C. glabrata: 108 spectra, C. krusei: 117 spectra, C. tropicalis: 128 spectra from five SDA plates each and Unknown: 90 spectra from nine SDA plates) were obtained from 20-25 different points on each sample's area of interest using Renishaw Wire 2.0 software.

Raman measurements were acquired using a Renishaw InVia Raman microscope (Renishaw, Gloucestershire, United Kingdom) by a 785 nm (infrared, IR) excitation laser with 50× objective. A single-grating spectrograph with a 1200 line/mm grating combined with a holographic notch filter for Rayleigh scattering rejection were used. A 50× objective lens was used to focus the excitation laser beam to a spot size of 4 μm×30 μm on a single colony within the SDA media. Once the laser was initiated, collection of the backscattered light took place with the laser power set at 100% (3 mW). Each spectrum consisted of the average of 2 collections with a 10 second collection time and an extended range of 600 to 1800 cm-1. Measurements were obtained using Renishaw Wire 2.0 software. At least 20 spectra were measured from different randomly selected points on each sample. The Raman Processing Software (RP Software) was used to import and analyze the data (Reisner et al., Chemometr Intell Lab Syst 2011; 105(1): 83-90). The acquired raw spectra were corrected by subtracting background fluorescence, reducing noise, and normalizing the intensities. Data were analyzed using Principal Component Analysis (PCA) and Discriminant Function Analysis (DFA). The peaks in Raman spectra (wavenumbers) that were most significant to the classification process were identified. The biochemical correlates of these Raman peaks were also described using previously published work De Gelder et al., J Raman Spectrosc 2007; 38(9): 1133-1147; Movasaghi et al., Appl Spectrosc Rev 2007; 42(5):493-541.

A databank or library of different Raman signatures for each of the five Candida species was created. Unknown colonies of Candida species were also derived using ATCC reference species (C. albicans, C. parapsilosis, C. glabrata, C. krusei and C. tropicalis) after creating the initial library databank. The investigating team was blinded to the identity of the unknown Candida species. Colonies from cultures of unknown Candida species, derived from sources other than those that were used to create the library, were compared with the known Raman signatures to identify their species.

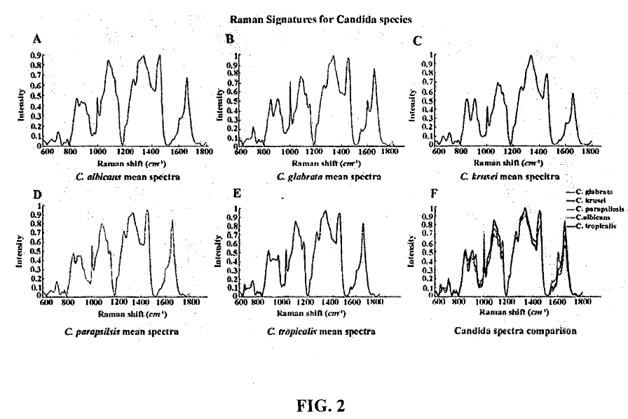

Results. When processed with RP Software, five Candida species examined showed eleven principal components generated by PCA, which account for 95.2 percent of the variance. Then the principal components were fed into DFA classifier, which enabled categorizing of unknown species. FIG. 2 represents the mean normalized curves for each species and demonstrates the key differentiators among them. The chemical structure of the fungal elements represents different peaks within the Raman spectra. Based on the known spectra, the trained DFA classifier was able to identify unknown sample signatures with 100% accuracy. These differentiating peaks for different biological molecules have been described De Gelder, supra; Movasaghi, supra. The Raman shift regions (wavenumbers/cm-1) associated with significant peaks within the Raman spectra and corresponding biochemical elements are described in Table 1.

TABLE 1
Wavenumbers of significant peaks in Raman spectra
and corresponding biochemical elements.

| Wave numbers (cm⁻¹) | Biochemical Elements |
|---|---|
| 649 | Amino acid |
| 716 | Nucleotide |
| 910 | Glucose |
| 1003 | Phenylalanine ring breathing mode |
| 1083 | Nucleic acid |
| 1152 | Protein/carotenoid |
| 1263 | Amide III band component protein |
| 1337 | Amide III/tryptophan/nucleic acid |
| 1452 | Delta CH2/Delta CH3 |
| 1603 | Phenylalanine |
| 1656 | Amide bonds |

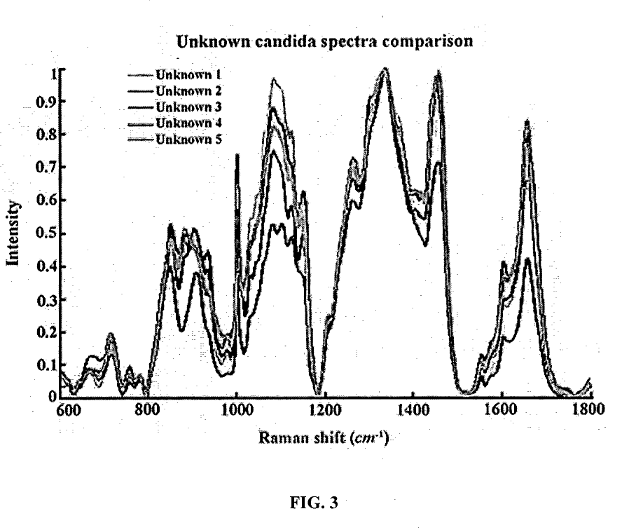

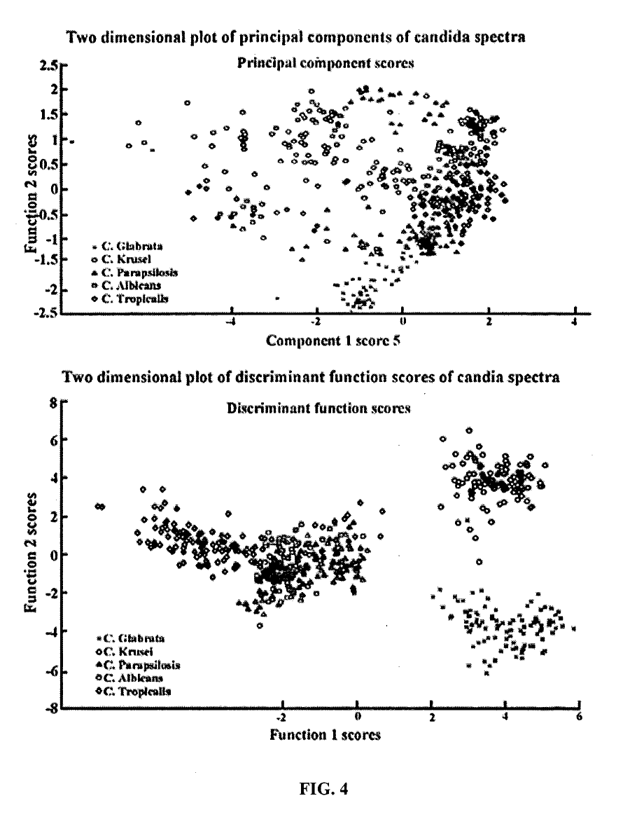

The Raman signatures for all the unknown species evaluated were compiled and placed together in FIG. 3. This figure again demonstrates key differences among the unknown samples. FIG. 4A represents two dimensional graphical representations of Principal Component 1 and Principal Component 2, and thus demonstrates the first step of PCA. Similarly FIG. 4B demonstrates two dimensional graphical representations of the first step of DFA.

In very low birth weight neonates with fungal BSIs, it is difficult at times to obtain blood samples for cultures and the amount of blood collected is sometimes suboptimal. This limits the sensitivity of blood cultures and some neonates with Candida infections might go undiagnosed until an autopsy is performed. Ahmad et al., J Clin Microbiol 2002; 40(7): 2483-2489. There are some non-culture methods for Candida identification including testing for fungal antigens and DNA available either commercially or in the evaluation phase. Mean, supra; Ahmad et al., Indian J Med Microbiol 2012; 30(3): 264-269. Use of DNA technology for detection of Candida has shown promise. Mean, supra. In very low birth weight neonates, however, the detection of fungal BSIs using DNA technology is not superior to fungal lysis-centrifugation isolator system. Trovato et al., Clin Microbiol Infect 2012; 18(3): E63-E5. The DNA technology requires use of costly reagents and technical expertise. The use of RS described herein did not include use of any consumables and evaluation was generally completed within 30 min per unknown sample.

This study is the first to demonstrate the use of RS for identification of Candida species recovered in cultures. After colonies grow on solid culture media, it takes approximately 48-72 hr to identify the Candida species. Morris et al., J Clin Microbiol 1996; 34(6): 1583-1585. The API 20C identification system used as a standard of practice involves carbohydrate assimilation by fungal colonies. RS involves identification of peaks of chemical elements followed by comparing those using PCA and DFA techniques.

RS is likely to reduce the identification time to only a few minutes because Raman signatures were useful in identifying the unknown Candida species with 100% percent confidence. This would be a significant advantage for very low birth weight neonates because they have limited ability to fight the invasive fungal infections. As demonstrated herein, RS is useful in decreasing time of identification of Candida species after the organism has grown from a positive culture. The methods disclosed herein can also be applied to use of Raman signatures for identification of organisms directly from clinical specimens.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. As used herein, a material effect would cause a statistically-significant reduction in ability to identify a Candida species using a method disclosed herein.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; +14% of the stated value; +13% of the stated value; ±12% of the stated value; ±11% of the stated value; +10% of the stated value; ±9% of the stated value; ±8% of the stated value; ±7% of the stated value; ±6% of the stated value; ±5% of the stated value; ±4% of the stated value; ±3% of the stated value; +2% of the stated value; or +1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

What is claimed is:

1. A method of identifying a fungal or bacterial species comprising: (a) establishing a database of characteristic curves of fungal or bacterial species; (b) performing a spectral analysis method on a sample obtained from a subject and obtaining corresponding characteristic curves; and (c) searching the database of step (a) for a match to the characteristic curves obtained in step (b), and (d) identifying a fungal or bacterial species in the sample.

2. A method of claim 1 wherein the establishing includes establishing characteristic curves for C. albicans, C. tropicalis, C. parapsilosis, C. kefyr, C. dubliniensis, C. lusitaniae, C. glabrata, C. krusei, and C. guilliermondi.

3. A method of claim 1 wherein the establishing includes establishing characteristic curves for C. albicans, C. parapsilosis, C. glabrata, C. krusei and C. tropicalis.

4. A method of claim 1 wherein the samples are obtained directly from a culture having a biological sample derived from the subject.

5. A method of claim 1 further comprising directing an antifungal or antibacterial treatment based on the identifying.

6. A method of claim 5 wherein the antifungal treatment is selected from fluconazole, caspofungin, anidulafungin, micafungin, voriconazole, amphotericin B deoxycholate, itraconazole or ketoconazole.

7. A method of claim 1 wherein the subject is a low birth weight neonate or an immunocomprised individual.

8. A method of claim 1 wherein the spectral analysis method is Raman spectroscopy or MALDI-TOF.

9. A method comprising testing a sample obtained from a subject suspected of suffering from a Candida infection for the presence of a spectral curve associated with a Candida species; assessing obtained spectral curves for matching to spectral curves of Candida species; identifying a Candida species based on the assessing, if a match is present.

10. A method of claim 9 wherein the identified Candida species is C. albicans, C. tropicalis, C. parapsilosis, C. kefyr, C. dubliniensis, C. lusitaniae, C. glabrata, C. krusei, or C. guilliermondi.

11. A method of claim 9 wherein the identified Candida species is C. albicans, C. parapsilosis, C. glabrata, C. krusei and C. tropicalis.

12. A method of claim 9 wherein the samples are obtained directly from a culture having a biological sample derived from the subject.

13. A method of claim 9 wherein the spectral curve is obtained using Raman spectroscopy or MALDI-TOF.

14. A method of claim 9 further comprising directing an antifungal treatment based on the identifying.

15. A method of claim 13 wherein the anti-fungal treatment is selected from fluconazole, caspofungin, anidulafungin, micafungin; voriconazole, amphotericin B deoxycholate, itraconazole or ketoconazole.

16. A method of claim 9 wherein the subject is a low birthweight neonate.

17. A method comprising obtaining a sample from a lowbirth weight neonate accumulating spectral data from the sample; identifying a spectral feature that is indicative of infection by a Candida species; directing a course of antifungal treatment based on the identifying.

18. A method of claim 17 wherein the Candida species is C. albicans, C. tropicalis, C. parapsilosis, C. kefyr, C. dubliniensis, C. lusitaniae, C. glabrata, C. krusei, or C. guilliermondi.

19. A method of claim 17 wherein the Candida species is C. albicans, C. parapsilosis, C. glabrata, C. krusei and C. tropicalis.

20. A method of claim 17 wherein the antifungal treatment is selected from fluconazole, caspofungin, anidulafungin, micafungin, voriconazole, amphotericin B deoxycholate, itraconazole or ketoconazole.

21. A method of claim 17 wherein the spectral data is obtained using Raman spectroscopy.
